(19) United States
(12) Patent Application Publication
(10) Pub. No.: US 2016/0193120 A1
(43) Pub. Date: Jul. 7, 2016

(54) HYDROXYL TERMINATED PDMS-BORATE ACID PUTTY AS DENTAL ISOLATION MATERIAL

(71) Applicant: 3M INNOVATIVE PROPERTIES COMPANY, Saint Paul, MN (US)

(72) Inventor: Bradley D. CRAIG, Lake Elmo, MN (US)

(21) Appl. No.: 14/916,078

(22) PCT Filed: Aug. 22, 2014
(86) PCT No.: PCT/US2014/052339

§ 371 Date: Mar. 2, 2016

Related U.S. Application Data
(60) Provisional application No. 61/873,006, filed on Sep. 3, 2013.

(51) Int. Cl. A61K 6/093

(52) U.S. Cl. CPC A61K 6/093

(57) ABSTRACT

The present application is directed to a method for isolating a working area in a patient's mouth comprising providing a moldable elastomeric composition, molding the composition to provide a moldable isolation device having a desired shape, making at least one perforation on the isolation device, and positioning the isolation device inside the patient's mouth to isolate the desired working area through the perforation. In such an embodiment, the moldable elastomeric composition comprises a polysiloxane composition.

### FIELD

The present application is directed to a composition, a method and a device for isolating tooth material to be treated and producing a shield for the surrounding gums and/or adjacent teeth.

### BACKGROUND

With some dental treatments, aggressive chemical substances are used in the mouth which can cause burning to the oral mucosa. For example, acid etching techniques at dental surgery, applying a composite filling, and bleaching with preparations containing a high percentage of peroxide. Furthermore, certain preparations and restorative techniques are sensitive to saliva, blood, and other fluid. It is therefore desirable to create a barrier to these fluids and leave the preparation area clean.

During the acid etching technique the dental enamel is treated with a highly-concentrated, generally about 35 wt. %, phosphoric acid within a prepared tooth cavity to improve the adhesion before the application of a primer and/or bonding. The phosphoric acid solution or phosphoric acid gel is then left to act for approximately 30 seconds on the dental enamel. When preparations lie in the vicinity of the gum or an adjacent tooth, it is necessary to isolate the treatment area and shield tissue from the etching means. It is further desirable to protect the treated tooth, or similarly a plurality of teeth, relative to the surrounding tissue of the oral cavity in a leak-tight fashion, to prevent, for example blood or saliva from reaching the treated tooth.

During a bleaching treatment, bleaching agents with a content of up to 35 wt. % hydrogen peroxide are directly applied onto the surface of the tooth to whiten the teeth. In some instances, the bleach is applied up to the vicinity of the gum margin. It is important to isolate the treatment area and protect the oral mucosa is also imperative in this case to prevent burning.

Currently, shielding is accomplished by using a resilient sheet made from rubber which is referred to as a dental dam. In its original and simplest form, it consists of an elastic, flat covering means, mostly in the form of a rubber cloth, which may be fastened in a frame outside the mouth. The dentist then has to perforate the sheet at suitable points and punch out holes of a corresponding size in the sheet, through which the teeth to be treated are subsequently pushed. Where the size of the holes is unsuitable or where there are imperfections on the surface of the tooth, the problem frequently arises that the resilient sheet does not sit and seal precisely or tightly enough along the gum margin and thus does not sufficiently protect the oral mucosa. The positioning of a dental dam is regarded by many dentists as too costly and awkward. The danger arises that when stretched or during the treatment the rubber tears or loosens. As a result the isolating effect of the dental dam is lost and the material present on the exterior of the rubber sheet can enter the oral cavity. Additionally, patients find the cumbersome equipment and the fastening of the rubber on the neck of the tooth unpleasant.

### SUMMARY

The present application is directed to a method for isolating a working area in a patient's mouth comprising providing a moldable elastomeric composition, molding the composition to provide a moldable isolation device having a desired shape, making at least one perforation on the isolation device, and positioning the isolation device inside the patient's mouth to isolate the desired working area through the perforation. In such an embodiment, the moldable elastomeric composition comprises a polysiloxane composition.

### DETAILED DESCRIPTION

The present application device is directed to utilizing a moldable elastomeric composition, for example hydroxyl-terminated PDMS (Polydimethyl siloxane) material reacted with a reversibly crosslinked material such as boric acid to create a formable, shapable, cuttable localized isolation material for use in dentistry. This material can also be repaired if torn, or the isolation hole is too large.

### Moldable Elastomeric Composition

### Polydimethyl Siloxane

Generally, the elastomeric composition is a polysiloxane composition.

In some specific embodiments, the polysiloxane comprises a polysiloxane material made by reacting hydroxyl-terminated polydimethyl siloxane (PDMS) with a borate or a boric acid. The borate/boric acid forms reversible crosslinks in the PDMS, which allows tears in the material to be easily healed by pressing the material back together and re-forming a seal.

The polydimethyl siloxane creates a barrier, which may assist in repelling moisture, blood, and other contaminants away from the isolated tooth area.

Generally, the hydroxyl-terminated PDMS is of the formula:

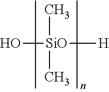

wherein n is an integer from 10 to 1500, or mixtures thereof. In some embodiments, n is an integer from 10 to 500, in specific embodiments, n is an integer between 10 and 100, for example between 25 and 80.

In some embodiments, the borate is of the formula:

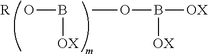

wherein m is an integer from 0 to 3, each R is independently chosen from hydrogen, monovalent hydrocarbon groups, and halogenated monovalent hydrocarbon groups, and each X is independently chosen from hydrogen, monovalent hydrocarbon groups, and halogenated monovalent hydrocarbon groups.
   In some embodiments, each R is independently chosen from hydrogen, aryl groups, halogenated aryl groups, aralkyl groups, aliphatic groups, haloaliphatic groups, and cycloaliphatic groups, and wherein each X is independently chosen from hydrogen, aryl groups, halogenated aryl groups, aralkyl groups, aliphatic groups, haloaliphatic groups, and cycloaliphatic groups. In specific embodiments, each R is independently chosen from hydrogen, phenyl, chlorophenyl, xylyl, tolyl, phenylethyl, benzyl, alkyl, alkenyl, halogenated alkyl, halogenated alkenyl, and cycloalkyl groups. In more specific embodiments, each R is independently chosen from hydrogen and lower alkyl groups.

In some embodiments of the borate, m is zero, each instance of X is hydrogen, and R is hydrogen.

In some embodiments. the polysiloxane composition comprises a polysiloxane material made by reacting hydroxyl-terminated PDMS with boric acid during a defined reaction time. In specific embodiments, the reaction time is in the range from 1 to 3 hr, for example 1.5 to 2.5 hr, in specific example 1.5 hours. The reaction may be carried out at a temperature from 130° C. to 220° C., for example 150° C. to 170° C. and in specific examples 170° C. In such an embodiment, the percent of boric acid may be from 2 to 9 weight percent with respect to the total weight of the reaction mixture, for example from 4 to 6 weight percent with respect to the total weight of the reaction mixture. In specific examples, the percent of boric acid is 5 weight percent with respect to the total weight of the reaction mixture. In some embodiments, the PDMS has a nominal molecular weight of 4200 g/mol.

### Additives

In some embodiments, additives may be incorporated into the elastomeric composition in order to modify the handling characteristics or adjust the patient experience. For example, thickening agents such as fumed silica may be added. Additional additives include colorants and flavorants.

### Use

Such an isolation material would potentially be simpler to use than existing rubber dental dams. The use of a moldable PDMS material crosslinked by boric acid potentially allows the dentist to isolate only a portion of the mouth. In some embodiments, the dentist may be able to avoid using a frame, though the isolation material of the present application may be used with any conventional frame.

Generally, the elastomeric composition is molded into a desired shape. Such molding may be done by hand or by machine. The isolation material may be carved, cut and shaped easily by hand.

In some embodiments, at least one perforation is made through the isolation device. Multiple perforations may be made, depending on the needs of the area to be isolated. The isolation device is the positioned inside the patient's mouth to isolate the desired working area through the perforation. In some instances, the edges of the isolation device surrounding the perforation may be pressed around the perforation into the tooth, hard tissue or soft tissue surrounding the area to be isolated, or the working area. The isolation device may also be stretched to conform the isolation device to the soft and/or hard tissue surrounding the working area. The perforation size may be enlarged or manipulated to be smaller, as needed for the working area.

The isolation material of the present application may also be more comfortable for the patient, and keeps the isolation to only the portion of the mouth that is being repaired.

The following examples further disclose embodiments of the invention.

### EXAMPLES

### Examples 1-8

### Designed Experiment

Cross linked hydroxyl terminated polydimethylsiloxane (PDMS) (25 g) was prepared by mixing boric acid (available from Alfa Aesar, Ward Hill, Mass.) and hydroxyl terminated PDMS (4200 MW available from Alfa Aesar, Ward Hill, Mass.) in a high shear mixture. The mixture was put into a 200 ml glass jar and heated in a convection oven. A series of cross linked hydroxyl terminated polydimethylsiloxane (PDMS) was prepared by an orthogonal designed experiment looking at variations in boric acid concentration (4 or 6 weight percent of the total mixture), temperature (150° C. or 170° C.) and reaction time (1.5 hours or 2.5 hours). The cross linked hydroxyl terminated PDMS was then qualitatively evaluated (on a scale of 1-10) for stretch, stiffness, tack and slump. Stretch was determined by pulling the material to the breaking point (10=stretch to fine string; 1=break immediately). Stiffness was determined by the degree of resistance the material showed when pressed with a finger (10=firm; 1=easily deformed). Tack was determined by gently pushing the material into a gloved hand and then pulling it back to see how much the material wanted to adhere to the hand (10=stuck aggressively; 1=no stick). Slump was determined by rolling the material into a ball (approximately 1 inch diameter), placing the ball in a petri dish then checking to see how much the material spread out after about 15 minutes (10=significant spread; 1=little discernible spread). The results are shown in Table 1 below.

TABLE 1

| | Wt % | | | | | | |
|---|---|---|---|---|---|---|---|
| | boric | Temp | Time | | | | |
| Example | acid | ° C. | (Hrs) | Stretch | Stiffness | Tack | Slump |
| 1 | 6 | 150 | 2.5 | 2 | 6 | 1 | 5 |
| 2 | 6 | 150 | 1.5 | 1 | 9 | 1 | 1 |
| 3 | 4 | 150 | 1.5 | 1 | 10 | 1 | 2 |
| 4 | 6 | 170 | 2.5 | 4 | 4 | 3 | 7 |
| 5 | 6 | 170 | 1.5 | 10 | 2 | 8 | 10 |
| 6 | 4 | 170 | 1.5 | 6 | 3 | 6 | 8 |
| 7 | 4 | 150 | 2.5 | 2 | 8 | 1 | 3 |
| 8 | 4 | 170 | 2.5 | 5 | 3 | 5 | 7 |

### Example 9

Cross linked hydroxyl terminated PDMS (50 g) was prepared by mixing 2.5 g of boric acid (5 wt %) with 47.5 g of PDMS (4200 MW) in a high shear mixer. The mixture was put into a 200 ml glass jar and heated in a convection oven at 170° C. for 1.5 hours. The resulting material was subsequently placed over a dental typodont and was successfully used to cover a series of artificial teeth. The material could be cut away from the artificial tooth intended to be isolated with a dental composite instrument and pushed into the interproximal spaces between the artificial teeth effectively sealing/isolating the tooth of interest from the surrounding environment.

Various modifications and alterations of the present invention will become apparent to those skilled in the art without departing from the spirit and scope of the invention.

In a first embodiment, this application is directed to a method for isolating a working area in a patient's mouth comprising providing a moldable elastomeric composition, molding the composition to provide a moldable isolation device having a desired shape, and positioning the isolation device inside the patient's mouth to isolate the desired working area. In such an embodiment, the moldable elastomeric composition comprises a polysiloxane composition.

In a second embodiment, this application is directed the method of the first embodiment wherein the moldable isolation device is molded by hand.

In a third embodiment, this application is directed the method of the first embodiment, further comprising pressing the edges of the isolation device around the working area to conform the isolation device to the soft and/or hard tissue surrounding the working area.

In a fourth embodiment, this application is directed to the method of any other embodiment, wherein molding the composition to provide a moldable isolation device having a desired shape is carried out by a method chosen from hand manipulation, instrument-aided, and a combination thereof.

In a fifth embodiment, this application is directed to the method of any other embodiment, further comprising stretching the isolation device to conform the isolation device to the soft and/or hard tissue surrounding the working area.

In an sixth embodiment, this application is directed to the method of any other embodiment, wherein the isolation device requires no hardware to maintain its place in the patient's mouth.

In a seventh embodiment, this application is directed to the method of any other embodiment, wherein the polysiloxane composition comprises a polysiloxane material made by reacting hydroxyl-terminated polydimethyl siloxane (PDMS) with a borate.

In an eighth embodiment, this application is directed to the method of any other embodiment, wherein the hydroxyl-terminated PDMS is of the formula:

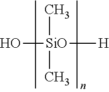

wherein n is an integer from 10 to 1500.

In a ninth embodiment, this application is directed to the method of any other embodiment, wherein borate is of the formula:

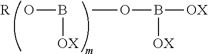

wherein m is an integer from 0 to 3, each R is independently chosen from hydrogen, monovalent hydrocarbon groups, and halogenated monovalent hydrocarbon groups, and each X is independently chosen from hydrogen, monovalent hydrocarbon groups, and halogenated monovalent hydrocarbon groups.

In a tenth embodiment, this application is directed to the method of any other embodiment, wherein the borate formula is defined further as boric acid.

In an eleventh embodiment, this application is directed to the method of any other embodiment, wherein the borate formula is defined further as each R is independently chosen from hydrogen, aryl groups, halogenated aryl groups, aralkyl groups, aliphatic groups, haloaliphatic groups, and cycloaliphatic groups, and
wherein each X is independently chosen from hydrogen, aryl groups, halogenated aryl groups, aralkyl groups, aliphatic groups, haloaliphatic groups, and cycloaliphatic groups.

In a twelfth embodiment, this application is directed to the method of any other embodiment, wherein the borate formula is defined further as each R is independently chosen from hydrogen, phenyl, chlorophenyl, xylyl, tolyl, phenylethyl, benzyl, alkyl, alkenyl, halogenated alkyl, halogenated alkenyl, and cycloalkyl groups.

In a thirteenth embodiment, this application is directed to the method of any other embodiment, wherein the borate formula is defined further as each R is independently chosen from hydrogen and lower alkyl groups.

In a fourteenth embodiment, this application is directed to the method of any other embodiment, wherein the borate formula is defined further as m is zero, each instance of X is hydrogen, and R is hydrogen.

In a fifteenth embodiment, this application is directed to the method of any other embodiment, wherein the polysiloxane composition comprises a polysiloxane material made by reacting hydroxyl-terminated polydimethyl siloxane (PDMS) of nominal molecular weight of 4200 g/mol with boric acid during a defined reaction time.

In a sixteenth embodiment, this application is directed to the method of any other embodiment, wherein the reaction time is in the range from 1 to 3 hr.

In a seventeenth embodiment, this application is directed to the method of any other embodiment, wherein the reaction was carried out at a temperature from 130° C. to 220° C.

In an eighteenth embodiment, this application is directed to the method of any other embodiment, wherein the percent of boric acid is from 2 to 9 weight percent with respect to the total weight of the reaction mixture.

In a nineteenth embodiment, this application is directed to the method of any other embodiment, wherein the reaction time is in the range from 1.5 to 2.5 hr.

In a twentieth embodiment, this application is directed to the method of any other embodiment, wherein the reaction time is 1.5 hr.

In a twenty-first embodiment, this application is directed to the method of any other embodiment, wherein the reaction was carried out at a temperature from 150° C. to 170° C.

In a twenty-second embodiment, this application is directed to the method of any other embodiment, wherein the reaction was carried out at a temperature of 170° C.

In a twenty-third embodiment, this application is directed to the method of any other embodiment, wherein the percent of boric acid is from 4 to 6 weight percent with respect to the total weight of the reaction mixture.

In a twenty-fourth embodiment, this application is directed to the method of any other embodiment, wherein the percent of boric acid is 5 weight percent with respect to the total weight of the reaction mixture.

In a twenty-fifth embodiment, this application is directed to a method for isolating a working area in a patient's mouth comprising: providing a moldable elastomeric composition, molding the composition to provide a moldable isolation device having a desired shape, and

positioning the isolation device inside the patient's mouth to isolate the desired working area, wherein the moldable elastomeric composition comprises a polysiloxane composition comprising a polysiloxane material made by reacting hydroxyl-terminated polydimethyl siloxane (PDMS) with a borate.

In a twenty-sixth embodiment, this application is directed to a method for isolating a working area in a patient's mouth comprising: providing a moldable elastomeric composition, molding the composition to provide a moldable isolation device having a desired shape; and making at least one perforation on the isolation device at a suitable position, positioning the isolation device inside the patient's mouth to isolate the desired working area through the perforation, wherein the moldable elastomeric composition comprises a polysiloxane composition comprising a polysiloxane material made by reacting hydroxyl-terminated polydimethyl siloxane (PDMS) with a borate.

In a twenty-seventh embodiment, this application is directed to a method for isolating a working area in a patient's mouth comprising: providing a moldable elastomeric composition; molding the composition to provide a moldable isolation device having a desired shape; making at least one perforation of a suitable size on the isolation device at a suitable position; and positioning the isolation device inside the patient's mouth to isolate the desired working area through the perforation, wherein the moldable elastomeric composition comprises a polysiloxane composition comprising a polysiloxane material made by reacting hydroxyl-terminated polydimethyl siloxane (PDMS) with a borate, the PDMS of the formula:

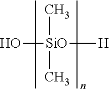

wherein n is an integer from 10 to 1500.

In a twenty-eighth embodiment, this application is directed to a method for isolating a working area in a patient's mouth comprising: providing a moldable elastomeric composition; molding the composition to provide a moldable isolation device having a desired shape; making at least one perforation of a suitable size on the isolation device at a suitable position; and positioning the isolation device inside the patient's mouth to isolate the desired working area through the perforation, wherein the moldable elastomeric composition comprises a polysiloxane composition comprising a polysiloxane material made by reacting hydroxyl-terminated polydimethyl siloxane (PDMS) of nominal molecular weight of 4200 g/mol with boric acid.

In a twenty-ninth embodiment, this application is directed to the method according to any preceding embodiments making at least one perforation on the isolation device, wherein the desired working area is within the perforation.

In a thirtieth embodiment, this application is directed to the method according to any preceding embodiments, wherein two or more perforations are made on the isolation device.

In a thirty-first embodiment, this application is directed to the method according to any preceding embodiments, further comprising enlarging or shrinking the size of the perforation on the isolation device to conform the isolation device to the soft and/or hard tissue surrounding the working area.

What is claimed is:

1. A method for isolating a working area in a patient's mouth comprising:
providing a moldable elastomeric composition;
molding the composition to provide a moldable isolation device having a desired shape; and
positioning the isolation device inside the patient's mouth to isolate the desired working area,
wherein the moldable elastomeric composition comprises a polysiloxane composition.

2. The method of claim 1 wherein the moldable isolation device is molded by hand.

3. The method according to claim 1, further comprising pressing the edges of the isolation device around the desired working area to conform the isolation device to the soft and/or hard tissue surrounding the working area.

4. The method according to claim 1, wherein molding the composition to provide a moldable isolation device having a desired shape is carried out by a method chosen from hand manipulation, instrument-aided, and a combination thereof.

5. The method according to claim 1, further comprising stretching the isolation device to conform the isolation device to the soft and/or hard tissue surrounding the working area.

6. The method according to claim 1, wherein the isolation device requires no hardware to maintain its place in the patient's mouth.

7. The method according to claim 1, wherein the polysiloxane composition comprises a polysiloxane material made by reacting hydroxyl-terminated polydimethyl siloxane (PDMS) with a borate.

8. The method according to claim 7, wherein the hydroxyl-terminated PDMS is of the formula: 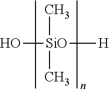
wherein n is an integer from 10 to 1500.

9. The method according to claim 8, wherein the borate is of the formula: 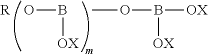
wherein m is an integer from 0 to 3, each R is independently chosen from hydrogen, monovalent hydrocarbon groups, and halogenated monovalent hydrocarbon groups, and each X is independently chosen from hydrogen, monovalent hydrocarbon groups, and halogenated monovalent hydrocarbon groups.

10. The method according to claim 9, wherein the borate is boric acid.

11. The method according to claim 9,
wherein each R is independently chosen from hydrogen, aryl groups, halogenated aryl groups, aralkyl groups, aliphatic groups, haloaliphatic groups, and cycloaliphatic groups, and wherein each X is independently chosen from hydrogen, aryl groups, halogenated aryl groups, aralkyl groups, aliphatic groups, haloaliphatic groups, and cycloaliphatic groups.

12. The method according to claim 9, wherein each R is independently chosen from hydrogen, phenyl, chlorophenyl, xylyl, tolyl, phenylethyl, benzyl, alkyl, alkenyl, halogenated alkyl, halogenated alkenyl, and cycloalkyl groups.

13. The method according to claim 9, wherein each R is independently chosen from hydrogen and lower alkyl groups.

14. The method according to claim 9, wherein m is zero, each instance of X is hydrogen, and R is hydrogen.

15. The method according to claim 9, wherein the polysiloxane composition comprises a polysiloxane material made by reacting hydroxyl-terminated polydimethyl siloxane (PDMS) of nominal molecular weight of 4200 g/mol with boric acid during a defined reaction time.

16. The method according to claim 15, wherein the reaction time is in the range from 1 to 3 hr.

17. The method according to claim 15, wherein the reaction was carried out at a temperature from 130° C. to 220° C.

18. The method according to claim 15, wherein the percent of boric acid is from 2 to 9 weight percent with respect to the total weight of the reaction mixture.

19. The method according to claim 15, wherein the reaction time is in the range from 1.5 to 2.5 hr.

20. The method according to claim 15, wherein the reaction time is 1.5 hr.

21. The method according to claim 15, wherein the reaction was carried out at a temperature from 150° C. to 170° C.

22. The method according to claim 15, wherein the reaction was carried out at a temperature of 170° C.

23. The method according to claim 15, wherein the percent of boric acid is from 4 to 6 weight percent with respect to the total weight of the reaction mixture.

24. The method according to claim 15, wherein the percent of boric acid is 5 weight percent with respect to the total weight of the reaction mixture.

25. A method for isolating a working area in a patient's mouth comprising:
providing a moldable elastomeric composition;
molding the composition to provide a moldable isolation device having a desired shape; and
positioning the isolation device inside the patient's mouth to isolate the desired working area,
wherein the moldable elastomeric composition comprises a polysiloxane composition comprising a polysiloxane material made by reacting hydroxyl-terminated polydimethyl siloxane (PDMS) with a borate.

26. A method for isolating a working area in a patient's mouth comprising:
providing a moldable elastomeric composition;
molding the composition to provide a moldable isolation device having a desired shape; and
positioning the isolation device inside the patient's mouth to isolate the desired working area,
wherein the moldable elastomeric composition comprises a polysiloxane composition comprising a polysiloxane material made by reacting hydroxyl-terminated polydimethyl siloxane (PDMS) with a borate.

27. A method for isolating a working area in a patient's mouth comprising:
providing a moldable elastomeric composition;
molding the composition to provide a moldable isolation device having a desired shape; and
positioning the isolation device inside the patient's mouth to isolate the desired working area,
wherein the moldable elastomeric composition comprises a polysiloxane composition comprising a polysiloxane material made by reacting hydroxyl-terminated polydimethyl siloxane (PDMS) with a borate, the PDMS of the formula: 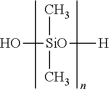
wherein n is an integer from 10 to 1500.

28. A method for isolating a working area in a patient's mouth comprising:
providing a moldable elastomeric composition;
molding the composition to provide a moldable isolation device having a desired shape; and
positioning the isolation device inside the patient's mouth to isolate the desired working area,
wherein the moldable elastomeric composition comprises a polysiloxane composition comprising a polysiloxane material made by reacting hydroxyl-terminated polydimethyl siloxane (PDMS) of nominal molecular weight of 4200 g/mol with boric acid.

29. (canceled)

30. The method according to claim 1 comprising making at least one perforation on the isolation device, wherein the desired working area is within the perforation.

31. The method according to claim 1, wherein two or more perforations are made on the isolation device.

32. The method according to claim 1, further comprising enlarging or shrinking the size of the perforation on the isolation device to conform the isolation device to the soft and/or hard tissue surrounding the working area.
